(19) United States
(12) Patent Application Publication
(10) Pub. No.: US 2011/0127448 A1
(43) Pub. Date: Jun. 2, 2011

(54) Treating Mixable Materials By Radiation

(76) Inventor: Eran Ben-Shmuel, Ganey Tiqva (IL)

(21) Appl. No.: 12/809,535

(22) PCT Filed: Dec. 3, 2008
(86) PCT No.: PCT/IL08/01572

§ 371 Date: Jan. 26, 2011

Related U.S. Application Data
(60) Provisional application No. 60/996,719, filed on Dec. 3, 2007.

(51) Int. Cl. G21K 5/02

(52) U.S. Cl. 250/492.1

(57) ABSTRACT

Provided are methods, systems, and radiating units for radiating mixable material, such as water, blood, sand, or the like, with sterilizing radiation. In one embodiment, the method includes immersing a radiation source in the mixable material, and moving the immersed radiation source inside the mixable material in a non regular fashion. The radiating unit optionally includes a radiation source; a power source for powering the radiation source; and a mobility agent that moves the radiating unit. The system optionally includes at least one radiating unit; and a mobility agent for moving the radiating unit.

### FIELD OF THE INVENTION

The present invention relates to a method and system for treating mixable materials by radiation, and to a radiating element suitable for use in such treatment. The present invention, in some embodiments thereof, is concerned with treatment of mixable materials, for example, water, blood, sand, and piles of wheat seeds, with radiation. An exemplary embodiment concerns sterilizing water with ultraviolet radiation.

### BACKGROUND OF THE INVENTION

UV (Ultraviolet) light is a known means for sterilization of blood and water. UV sterilization is sometimes advantageous over heat sterilization because UV sterilization does not necessitate substantial heating of the treated material. Some UV sterilization methods are advantageous over chemical sterilization in that no addition of active chemical agents is necessitated by the UV sterilization.

US 2006-144771 describes a filtering and purifying system, wherein a transparent glass tube is immersed into pretreated water in a stirring tank, and a sterilizing ultraviolet lamp is inserted into the glass tube to apply ultraviolet rays into the water. The pretreated water is moved all over to the vicinity of the ultraviolet lamp by a stirring blade and hence, the ultraviolet lamp having a weaker illumination intensity and a smaller power consumption can be used, leading to an effects of reducing the lamp cost and a sterilizing operational cost. The system uses photo synthesizers, and the UV light is used for synthesizing them.

EP 1,289,991 describes a system for inactivation of microorganisms in a blood sample held in bags. An agitator provides oscillatory motion to the bag. A pin or other structure is placed across or within the bag to provide turbulent eddies in the treated fluid. The agitator may be connected to a computer or other controller to control or monitor temperature of the fluid, energy output of the lights, agitation motion, and timing. The light source and fluid being treated may both move to provide agitation of the fluid, or only the fluid being treated may move while the light source remains stationary.

WO03/037504 describes a vibratory stirrer comprising vibration blades fixed to a vibration transmitting rod vibrating while being linked with a vibrating motor. Each vibration blade has a surface made of anatase-type TiO2activated by a photo catalyst.

UV-rays emitted from a light source are guided by a light guide to optical fibers, held to extend along the surface of the vibration blades. Each of the fibers has a light-leaking part for leaking light to the surface of the vibration blade. An enclosed waterproof illuminator emitting ultraviolet radiation can be used as the light emitting member of the light irradiating means. The abstract states that even a liquid having a low light transmittance can be sterilized with the system in a short time without requiring any chemical.

Other publications that may serve to understand the background of the invention include U.S. Pat. No. 5,780,860; U.S. Pat. No. 6,113,566; US 2006-0183987; WO 2006/096827; UA 9798 U; U.S. Pat. No. 5,536,395; WO 2005/090782; US 2003-201160; and U.S. Pat. No. 5,308,505.

### SUMMARY OF THE INVENTION

As indicated above, it is generally known to use UV radiation for sterilization of blood and water. However, the conventional UV sterilization techniques suffer form the limited UV light penetration depth, which sometimes is very small, for example 25 microns in blood and 1 cm in clean water. Therefore, with conventional UV sterilization techniques, UV sterilization is sometimes slower than heating sterilization or chemical sterilization.

An aspect of some embodiments of the invention concerns a novel technique for treating mixable materials using radiation.

Examples of mixable materials include fluids, for instance, water; suspensions, for instance blood; powders, for instance, sand; and piles of small particles, for instance wheat seeds.

Direct interaction (contact) of a radiating element with the mixable material maximizes the amount of effective radiation that reaches the material per unit time for a given radiation source. Therefore, a radiation element that is immersed in the mixable material, rather than irradiating it from the outside, makes more efficient use of the radiation energy.

In an exemplary embodiment, a method of treating mixable material comprises:

immersing a radiation source in the mixable material;

enabling relative displacement between the radiation source and the mixable material; and

radiating the mixable material from the radiation source in order to achieve treatment.

Preferably, the relative displacement between the radiation source and the mixable material is carried out in a random (irregular) fashion.

Preferably, the radiation source is moving inside the mixable material.

Optionally, the radiation source is free to move within the treated material, without being mechanically attached to an external power source or mobility agent.

Optionally, the movement of the radiation source is irregular.

Optionally, the movement of the radiation source causes movement in the mixable materials. For example, in one embodiment, radiation sources are moving inside a pool of standing water to sterilize the water, and while doing so create waves in the water.

Optionally, movement of the mixable material causes movement of the radiation source. For example, in one embodiment, radiation sources are immersed in a half-filled water bottle, and moved by agitating the bottle.

An aspect of some embodiments of the invention concerns an apparatus for treating mixable material using radiation. In an exemplary embodiment the apparatus comprises: a radiation source, associated with a radiating element. The radiating element is optionally configured to allow immersing the radiation source in the mixable material, in a way enabling relative motion between the radiation source and the mixable material. Optionally, the relative motion is random. In some embodiments, the apparatus also includes a mobility agent, configured to move the radiating element inside the mixable material.

Hereinafter, UV light is referred to as the radiation by way of example, however, the invention concerns any kind of radiation, including such radiation the effect of which decays within a short distance from the radiation source, for example, γ radiation.

Thus, there is provided in accordance with an exemplary embodiment of the invention, a method of irradiating mixable material with radiation, the method comprising:

immersing a source of said radiation in the mixable material; and

moving the immersed radiation source inside the mixable material in a non regular fashion.

Optionally, moving the immersed radiation source comprises powering movement of the radiation source by a power source that moves with the radiation source.

In some exemplary embodiments, the radiation source is mechanically attached to a moving member, and moving the immersed radiation source comprises moving said member.

Optionally, moving said member is from outside said mixable material.

The random (or quasi-random) fashion may be provided by random (independent) variation of the movement parameter(s) in space and time. For example, a plurality of movements may be created, each having a direction, wherein the direction of each of said movements is independent of the direction of the preceding movement; or each being along a distance, and the distance of each of said movements is independent of the distance of the preceding movement.

Optionally, the mobility agent comprises a element, (for example, a paramagnetic element) associated with an external magnetic field source, which creates a non regularly changing magnetic field, thus moving the radiation source in a non regular fashion.

According to one exemplary embodiment of the invention, the method comprises immersing a plurality of radiation sources in the mixable material; and moving each of said plurality of radiation sources inside the mixable material in a non-regular (e.g. random, quasi-random) fashion.

Optionally, the distance between each two of said plurality of radiation sources changes in time.

Alternatively or additionally, the distance between each two of said plurality of radiation sources changes in time in a quasi-random fashion.

Still alternatively or additionally, the distance between each two of said plurality of radiation sources changes in time in a non-periodic fashion.

In an exemplary embodiment, the radiation source may be the so-called “active source”, namely requiring a power supply to effect radiation. In this case, a power source may move with the radiation source or may be stationarily mounted outside the treatment zone and operate the radiation source via fiber(s) or wireless signal transmission. In another exemplary embodiment, the radiation source is a “passive” source which does not require powering (e.g. radioactive source).

There is also provided in accordance with an exemplary embodiment of the invention a radiating element comprising: a radiation source; and a mobility agent that moves the radiating element, optionally in a non-regular fashion.

Optionally, the radiating element comprises a first power source for powering said radiation source; and possibly also a second power source for powering said mobility agent.

Optionally, the first and second power sources are the same.

Optionally, the radiation source is encased in a waterproof casing.

Optionally, at least one of said first and second power sources is encased in a casing.

Optionally, the radiation source; and the power source(s) are all encased in a same casing.

Optionally, said same casing is waterproof.

In an exemplary embodiment, the mobility agent moves said radiation source in a quasi-random fashion.

In an exemplary embodiment, the radiating element is surrounded by a mixable material, and is in mechanical contact only with said mixable material.

Optionally, the radiation source comprises a UV radiation source.

Optionally, the radiation source comprises a LED.

In an exemplary embodiment, the radiating element comprises a processor that generates pseudo-random numbers and uses said numbers for controlling the movement of the radiating element.

There is also provided in accordance with an exemplary embodiment of the invention a radiating element comprising:
a passive radiation source, and
a mobility agent for moving the radiating element.

Optionally, the radiation source comprises a radioactive radiation source.

Optionally, the case encases a power source for powering said mobility agent.

Preferably, the mobility agent moves said radiation source in a quasi-random fashion. Optionally, the radiating element comprises a processor that generates pseudo-random numbers and uses said numbers for controlling the movement of the radiating element.

There is also provided in accordance with an exemplary embodiment of the invention a system for treating a mixable material, the system comprising:

at least one radiating element; and

a mobility agent for moving the radiating element, wherein each of said at least one radiating element comprises:
a radiation source; and
a power source for powering said radiation source.

Optionally, the mobility agent is capable of moving the radiating element when the radiating element is in continuous mechanical contact only with the mixable material.

Optionally, the radiating element is as described above.

In an exemplary embodiment, the system comprises a field generator for coupling a force field with said radiating element.

Optionally, the system comprises a container for holding the mixable material.

Optionally, the field generator is external to said container.

Optionally, the system comprises a separator for separating the at least one radiating element from the mixable material.

In a preferred embodiment, the system comprises a plurality of radiating elements. Optionally, the distance between each two of said radiating elements changes in a quasi-random fashion.

Optionally, the system comprises a container for holding the mixable material. Optionally, the mobility agent moves the at least one radiating element inside the container. Optionally, the mobility agent is entirely inside the container.

Optionally, the mobility agent is entirely inside the radiating element.

Optionally, the container has a volume that is at least 10 times larger than the volume of all said at least one radiating elements together.

In an exemplary embodiment, the system does not include a means for moving the mixable material.

### DETAILED DESCRIPTION OF EMBODIMENTS

### Overview

The present invention, in some embodiments thereof is concerned with treatment of mixable materials with radiation.

According to certain embodiments of the invention, radiation is applied to a mixable material by one or more radiating elements. Optionally, the radiating element is relatively mobile in the mixable material.

The term relatively mobile element refers herein to an element that can move in the mixable material, to an element that the mixable material can move around, or to an element which is both capable of moving in the mixable material and the mixable material can move around. Optionally, a relatively mobile element is static in relation to a container containing the mixable material.

As used herein, mobility can be active, passive, or both.

Active mobility is optionally achieved by a mobility agent, configured for generating relative mobility of the radiating element in the mixable material. For example, in one embodiment, a radiation source associated with a magnetic body is immersed (wholly or partially) in water, and rotated using a magnetic stirrer. Operating the stirrer in this case generates active relative mobility of the magnetic body in the water, and the radiation source is actively mobile in the water.

In the latter exemplary embodiment, the mobility agent comprises the magnetic body associated with the radiation source, which is immersed in the mixable liquid and the stirrer, which is optionally external to the mixable material.

Passive relative mobility of the radiating elements is achieved due to movement (such as flow) of the mixable material that is not intended for that purpose. For example, when a human carries a bottle of water, the walking movements of the human move or vibrate the bottle so as to mix the water inside the bottle. If a radiating element is immersed in the water, the walking movements result in relative mobility of the radiating element in the water. The relative mobility in this case is not intentional and hence is considered hereinafter as passive mobility.

In some embodiments, a mobility agent comprises a coupling member, coupling the radiating element to a moving mechanism. One example of a coupling member is a magnetic body coupling the radiating body to a stirrer. Another example of a coupling member is a chord, coupling the radiating element to a rotor.

Hereinafter, “association” of a radiating element with a body may mean, for example, that the body is the radiating element, that the body includes the radiating elements, and/or that the body is attached to the radiating element.

In some embodiments, the radiating element is associated with a non-radiating structure, possibly of a different purpose and material. Optionally, the non-radiating structure is static. Alternatively, the non-radiating structure is moving. For example, when materials are regularly stirred, for instance, granita is often stored in kiosks with constant stirring. Associating a radiating element to the stirring blades of a granita container optionally serves to produce sterilized granita.

In exemplary embodiments, radiation is powered by a power source, referred herein as a first power source. Optionally, the power source is internal to the treated material, preferably moving together with the radiating element. In one exemplary embodiment, the power source and the radiating element reside together in a waterproof capsule. The waterproof capsule is at least partially transparent to UV light.

Additionally to the first power source, a second power source is optionally used for enabling relative mobility. In active relative mobility, the second power source is optionally associated with the radiating element, while in passive mobility the second power source is optionally external to the radiating element, for example, the walking human carrying a bottle, as described above.

In some exemplary embodiments, a single power source makes both the first and second power sources. For example, in active mobility, a single battery can be used for radiation and for mobility. Optionally, the single power source is associated with the radiating element. For instance, the radiating element and the single power source are both encased in the same casing, such that in operation they move together in the mixable material.

Optionally, the first and second power sources are independent of each other.

Optionally, the first and second power sources depend on each other.

According to an exemplary embodiment, the movement of the radiating unit is used for charging the first power source, which powers the radiation source. In particular, this is useful when the mobility is passive. For example, when the movement is caused by the walking movement of a person carrying a bottle with liquid and radiating units, the movement created by the person is optionally used also for powering the first power source, for example, utilizing a kinetic generator or charger.

### Exemplary Shapes of Radiating Elements

In some embodiments, as the effective surface of a radiating element is larger the volume treated by the element is larger.

A radiating element is optionally two, two-and-a-half, or three dimensional (2-D, 2.5-D, or 3-D respectively). Some considerations in selecting a radiating element of a certain dimensionality to a given application include: the hydrodynamic requirements from the radiating unit, the number and shape of parts to be associated with the radiating unit (for example, the shape of available radiation source, the first power source, the mobility agent, the second power source, etc), and the desired surface to volume ratio. For example, 3-D units allow most flexible arrangements of parts, while 2D units have largest surface to volume ratio.

An element is considered 3-D if all its three dimensions differ from one another in no more than factor of 5.

An element is considered 2.5-D if it has two dimensions, each of which is larger than a third dimension, in a factor of between 5 and 50.

An element is considered 2-D if it has two dimensions, each of which is larger than a third dimension, in a factor larger than 50.

According to some embodiments of the invention, 3-D and 2-D elements are designed such that the radiation is emitted from the entire surface area of the element. One such design is a toroidal element, having a radiating ring encased in a toroidal case.

According to certain embodiments, increase of the effective surface area of a radiating element causes an increase in the volume of the mixable material exposed to the radiation of the radiating element, and hence decreases the process time, required to achieve satisfactory level of treatment.

The 3-D or 2-D element's shape influences the flow dynamics of the element in the treated medium and is optionally fine-tuned to it. In one example, for treating liquids, an ellipsoidal shape is optionally preferred over a spherical shape for achieving better liquid dynamics. In another example, toroidal shapes of larger inner rim are optionally preferred for treating materials with greater penetration depth.

### Power Source

According to some embodiments of the invention, the first power source (e.g. battery) is joined to the radiating element, for instance, by being cased together with the radiation source inside the radiating element, and powers the radiation of the radiating element (e.g. UV LEDs (light emitting diodes)).

Optionally, the first and/or the second power source are rechargeable. In some embodiments a rechargeable power source may be recharged, for instance, between consequent uses of the system. In some embodiments, a rechargeable power source utilizes an internal charger residing in the radiating element, for example, a kinetic charger; and/or a charger external to the element. An example of an external charger is socket connected to the general electricity mains. Another example to an external charger is a magnetic charger, as used in some commercial automatic tooth brushes. Docking to an external charger optionally involves direct contact between the charger and the power source. In some options, docking does not involve such direct contact.

According to some embodiments of the invention, the first and/or second power source are directly coupled to the radiating element and/or to the mobility agent, respectively, e.g., through wires. The wires are preferably arranged not to entangle when the radiating element moves around in the mixable material. Optionally, the power sources are connected to their respective power consumers via short and rigid connectors. Alternatively or additionally, the power consumers are connected to their respective power sources via dynamic connector, for example, rails.

### Control

According to some embodiments of the invention, various characteristics of the radiating element are controllable during operation. Examples of controllable characteristics include velocity inside the mixable material, moving direction in the mixable material, radiation intensity, the shape of the element and the radiation surface area.

In some embodiments, control is done via wired communication, wireless communication, and/or by internal logic. In some embodiments where power wires go into the radiating element from an external power source, one or more of the power wires are used as communication wires, for instance, for controlling the treatment.

According to some embodiments of the invention, different characteristics of the treatment may be controlled, manually or automatically, as function of one or more predetermined parameters, for example, process time, (which is the time between treatment beginning and end, including possible interruptions), and treatment time (which is the total time the material is treated, excluding interruptions, if exist).

In some embodiments, a first radiation intensity is used for some time, and then, radiation intensity is changed. Optionally, the radiation intensity is maintained at some predetermined range or value until satisfactory level of treatment is achieved, and then decreased, optionally to zero.

In some embodiments, decreasing the radiation intensity to levels above zero after satisfactory treatment level has been achieved prevents the treatment level from deteriorating to unacceptable level. The term treatment level parameter is used below to refer to one or more parameters, quantifying or qualifying the treatment result. Examples of treatment level parameters include bacterial volumetric density, protein volumetric density, electric conductivity, temperature, and optical transparency.

In some embodiments, temporary values of one or more treatment level parameters are determined during operation. Optionally, treatment parameters, such as the radiation intensity or movement characteristics are changed in response to determined treatment level parameters. For example, in one embodiment, if one of the treatment level parameters changes at the beginning of the treatment, and stops changing before it reaches a satisfactory level, the radiation intensity is increased.

In some embodiments, the process time expected to be required for achieving satisfactory treatment is predetermined, and the treatment is programmed in accordance with this time, for instance, that after the predetermined time treatment stops. Predetermining the process time is optionally by theoretical computation and/or by computer simulation.

In the following, the term sterilization is used instead of “treatment that achieves satisfactory treatment level”, and is not necessarily relates to a biological effect of the treatment.

According to some embodiments of the invention, satisfactory treatment requires destruction of certain materials in the mixable material. Examples of materials that are to be destroyed in some embodiments are proteins and genetic substance, for instance, nucleic acids.

In some embodiments, satisfactory treatment level is achieved when the genetic substance of a virus is totally destroyed. These embodiments require relatively long treatment periods and/or relatively high intensity.

In some other embodiments, total destruction is not required, and satisfactory treatment level is associated with prevention of virus reproduction. In such embodiments, shorter treatment time and/or lower radiation intensity may be sufficient.

According to certain embodiments treatment includes chemical or physical modification of the mixable material or a component thereof other than that provided by the radiation. Examples to such modifications include: heating, cooling, and/or addition of chemical agents, for example, chlorine, chlorine dioxide, and/or ozone. Alternatively or additionally, the sought modification comprises physical modification, for example, modification of a substance tertiary structure (e.g., the tertiary structure of a protein).

According to some embodiments of the invention, characteristics which define quality of the treatment results are measured during the process and the results transmitted continuously or in a timed manner to a controller. In some embodiments, the controller is external to the container holding the mixable material. Alternatively or additionally control is with an external controller. In some embodiments, an internal controller, associated with a radiating element, communicates with an external controller to provide the external controller data gathered by instrumentation mounted on the radiating unit, and to receive from the external controller control signals. Optionally, the external control processes signals received from a plurality of radiating units, decides on a treatment scheme based on the received signals, and communicates control signals to the radiating units in accordance with the decided treatment scheme. In some embodiments, radiation units communicate directly for each other. For instance, in a certain embodiment, each radiating element has a detector for detecting the contamination level at the vicinity of the element, and if this level is above a predetermined value, transmits signals indicative to the high contamination level. The units receiving the signal optionally direct their motion towards the signaling unit, so as to provide more intense treatment in the more contaminated region.

A measurement of a treatment level parameter is optionally taken from within the container or from outside the container. For example, in some embodiments, the container is transparent, and light absorption of the mixable material is measured with a light source illuminating the mixable material through a transparent container. Optionally, the detector, used for detecting the light, after interaction with the mixable material is also external to the container.

In some embodiments, a sensor is associated with the radiating element. Optionally, sensors associated with a radiating element are used for sensing conditions inside the mixable material. Optionally, the sensor is immersed (wholly or partially) in the mixable material.

A sensor associated with the radiating element is optionally used to provide data on characteristics of the mixable material as the material is being treated. Some such sensors optionally comprise miniature temperature sensors, miniature LEDs (Light emitting Diodes), miniature light detectors, for example, niniature cameras, miniature chemical analyzers (for example, for CO₂ detection, and/or salinity evaluation), velocity detectors and accelerometers. Such components are optionally arranged to measure the viscosity of the mixable material.

In some embodiments, planning an effective radiation treatment according to the invention, is based on an estimation of the minimal radiation power that is effective for sterilization (hereinafter “minimal effective radiation power”). Some parameters affecting the minimal effective radiation power include the radiating element's structure, the container structure, the number of radiating elements immersed in the container, the radiation source efficiency, the amount of mixable material in the container, and the treatment level that is considered satisfactory.

For example, different applications require different treatment levels, and therefore, may require different treatment powers. For instance, in one embodiment, 3 Watts of radiated energy are required to totally destroy a certain virus within 1 minute, while 2 Watts radiated during the same time are required to effectively change the virus' genetic material's tertiary structure. In those cases when sterilization can be satisfied with prevention of reproduction, it is appreciated that 2 Watts radiated during 1 minute is enough.

Further to defining the required minimal effective radiation intensity, the “useful volume” of a radiating element can be defined. Hereinafter “useful volume” in a certain mixable material is a function of effective radiating element's surface area, the penetration depth, and the radiation intensity. The effective radiating element's surface area is the surface area from which the radiating element is radiating. Those versed in the art would appreciate that radiation intensity is not necessarily uniform throughout the surface area, or in different words, the radiation intensity may change as a function of the coordinates on the effective area.

The penetration depth is characterizing the mixable material wherein the radiating element is immersed and the wavelength of the radiation. The penetration depth is optionally known in advance, and taken into consideration in designing a sterilization system for a certain application. Alternatively or additionally, the penetration depth of a given radiation in a given mixable material is measured online or offline, as known per se.

The radiation intensity is optionally equal to or larger than the minimal effective radiation intensity. Appreciating that radiation may sometimes damage the mixable material (“secondary destruction”, which may be thermal or other), it may be useful to keep the radiation intensity below the level effecting secondary destruction. The radiation intensity that causes secondary destruction is influenced by the relative movement of the radiating element in the mixable material: a moving element can radiate in higher intensity than a stationary (immobile) similar element without causing secondary destruction, and the higher the element's velocity is, the higher is the radiation required to cause secondary destruction.

Moving the radiation element in the mixable material provides more rapid exchange of material around the radiating element than is provided with a stationary element. Thus, more material is exposed to radiation, even if for shorter periods, when there is relative motion between the radiating element and the mixable material. The faster is the movement of the element, there is less room to undesired local effects of the radiation, such as overheating, or saturation (that is, radiating the same molecules or particles after satisfactory treatment level is achieved). In some embodiments, faster motion is associated, with higher radiation power to compensate for shorter exposure periods.

In some embodiments, the treatment results are not cumulative. For example, in some applications, short exposure to radiation within acceptable intensity limit is not effective, while longer continuous exposure to radiation of similar intensity is effective. In such a case, many short radiation periods are useless, while longer exposure periods (associated with slower movement) provide effective sterilization.

In some embodiments, a very short exposure time is sufficient, and exposing the material to longer periods is a waste.

Furthermore, viscosity of the mixable material also affects the amount of mixable material that is exposed to the useful volume under given radiation conditions. Theoretically, the lower the viscosity, the higher is the amount exposed to the useful volume in certain time duration, while the effect is not necessarily linear.

When the radiating element is moving, the mixable material in the useful volume, changes. In a low viscosity mixable material, such as water, most of the exposed mixable material changes, while in higher speeds higher change rate usually occur. In some cases, therefore, higher speeds are associated with shorter treatment time.

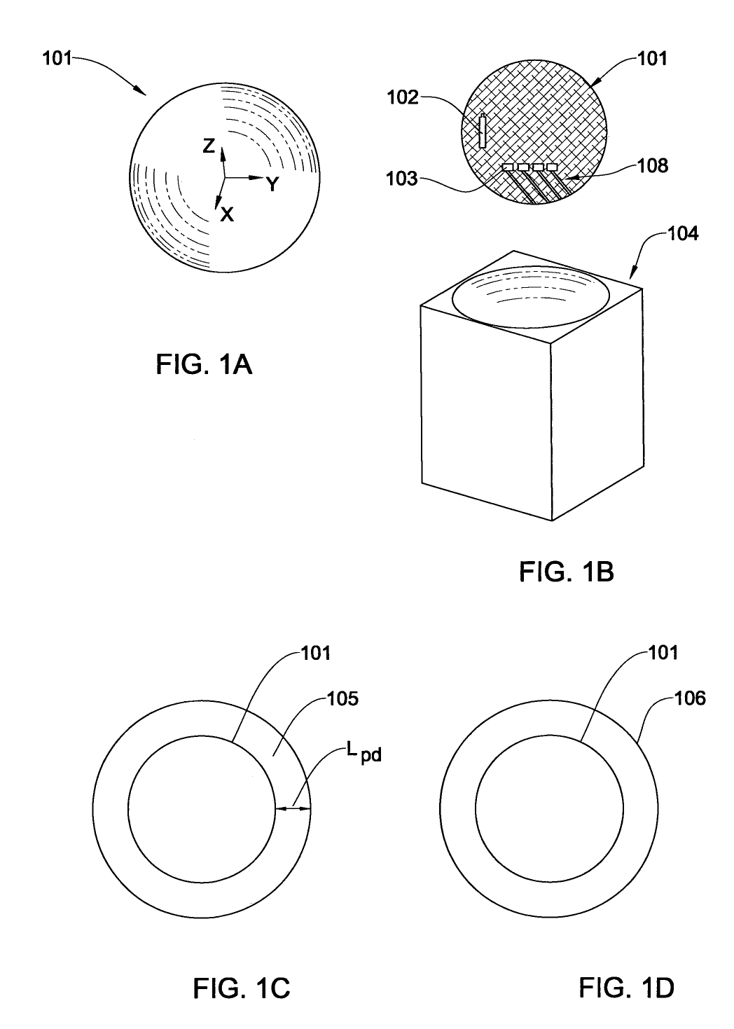

FIG. 1A illustrates a radiating element 101 having a form of a sphere, according to one embodiment of the invention. In one example, the sphere's radius is 1 centimeter (cm). According to certain embodiments, the sphere contains a battery 102 and one or more UV LEDs 103 inside, as illustrated in FIG. 1B. According to the depicted embodiment, each one of the LEDs 103 is a radiation source. In addition, the battery 102 is a power source that allows activating the radiation sources (the LEDs) for radiating the mixable material in order to achieve treatment. The sphere 101 is an example of a radiating element that can be immersed, and therefore allows immersing the radiation sources, (LEDs) in the mixable material.

In FIGS. 1A and 1B the entire surface of the radiating element 101 is UV transparent (in this example the element's surface is made of a material transparent to UVA, UVB and UVC, such as quarts), and hence the entire surface of the sphere emits light in certain wavelengths and intensities provided by the LED.

In some embodiments, battery 102 is rechargeable upon docking the sphere to a docking station 104. Optionally, the LEDS are turned off during docking.

The active area of the sphere of FIG. 1 is: S=4πr²=4π·1²=4π[cm²].

Optionally, the sphere moves each second a distance equal to the sphere diameter; in the above example, this is 2 [cm/sec].

In a non-viscous mixable material, the material making the useful volume is replaced in such a pace, that all the material is replaced when the sphere traveled a distance equal to the sphere's radius.

The penetration depth is marked, hereinafter, as L_{pd}.

According to the present example, the penetration depth of UV in the mixable material is L_{pd}=25 [μm]=2.5E−3 [cm]. A layer having a width of L_{pd}, coating the radiating element, is illustrated as layer 105 in FIG. 1C. Appreciating that the radiating element is 3D (3 dimensional) and the entire surface area is UV transparent, The volume of layer 105 can be estimated as a volume of a spherical shell, 2 cm in diameter and 25 microns in thickness.

The volume of the spherical shell around the sphere is: V≈S·L_{pd}=4π·2.5E−3=31.42E−3 [cm³].

Therefore, given half a liter of mixable material and assuming that 5 spheres are immersed in the mixable material, wherein the minimal effective radiation intensity is 1 Watt radiated during 1 second, it will take

$$\frac{500}{5 \cdot 31.42 E - 3} = 3182.686 [ sec ] = 53 [ min ]$$

to complete a radiation treatment (e.g. sterilization).

By replacing the 1 [cm] spheres with spheres having a radius of 2 [cm], keeping all other parameters (the speed and the number of spheres, and the radiation intensity at the surface of the sphere) without a change, it is possible to complete the same radiation treatment within only 44 minutes.

By altering additional parameters, such as the number of spheres, the speed of movement, and the radiation intensity at the surface an even shorter radiation treatment can be achieved. For example, using three spheres, each 2 [cm] in radius moving at 20 [cm/sec], and irradiating in 5 times greater intensity will take 4.42 [min] to complete the same treatment to the same amount of mixable material. This number is calculated using the following parameters:

| | | |
|---|---|---|
| Lpd | 25.000 | [um] |
| number of spheres | 3.000 | |
| velocity | 20.000 | [cm/sec] |
| radius | 2.000 | [cm] |
| liquid volume | 500.000 | [cm\hat{ }3] |
| active area | 50.265 | [cm\hat{ }2] |
| active volume | 0.126 | |
| sterilization time[sec] | 265.258 | |
| sterilization time[min] | 4.421 | |

The examples illustrated so far, with reference to FIGS. 1A to 1C are non-limiting. According to different embodiments, instead of using LEDs as the radiation source, other radiation sources can be used. For example, a bulb emitting any wavelength applicable to the case, including UV, IR (Infrared) and even a spectrum of wavelength. According to a different embodiment it is possible to use non-stable materials (such as radioactive materials), whose nuclear fission is accompanied by emitting radiation. It should be appreciated that in embodiments wherein such materials are used as the radiation source, a first power source is not necessarily required. If the emitted radiation intensity is high enough to achieve the required sterilization treatment, a power source for powering the radiation source can be omitted.

### Controlling the Trajectory

Optionally, each radiating unit moves in the mixable material independently of the other units (except for some occasions when two units collide or the like).

Optionally, the movement of each radiating unit depends on the movement and/or position of the other units, for instance, in a manner preventing collisions between two radiating units.

Optionally, the movement of the radiating units is at constant rate and direction until a collision occurs. Optionally, the collision is with the container wall. Optionally, the collision is with another radiating element. Optionally, when a collision occurs, the direction of the colliding unit changes due to the collision, and constant movement continues. Optionally, after a collision, the movement direction changes in a predetermined angle, for example, 30°, 90°, 180°.

Optionally, the direction at which a radiating unit moves changes periodically, for example, every 10 seconds. Optionally, each time, a movement direction is chosen randomly. Optionally, each time the movement direction changes in a predetermined angle.

Optionally, some steps are longer than others. For examples, each step of 20 seconds in a predetermined direction is followed by 10 steps of 5 seconds each in a different direction.

### Protecting the Element

Sometimes the radiating element may be vulnerable to damage caused by the medium in which it is immersed, such as the mixable material or the conditions required to keep the mixable material etc. For example, the temperature of liquid Nitrogen (at atmospheric pressure) is −196° C. Therefore, a radiating element according to certain embodiments of the invention intended for sterilizing liquid Nitrogen optionally comprises athermal coating. Preferably, the thermal coating does not interfere with the radiation.

Returning to the examples illustrated in FIGS. 1A-1C, in some embodiments sphere 101 is coated with a quartz coating layer 106, defining a vacuum layer 107 separating between the sphere's outer surface and quarts layer 106.

Coating layer 106 is UV transparent (as it is made of quartz) and the vacuum layer is also UV transparent, and the radiation emitted from LEDs 103 will reach the mixable material outside coating layer 106. Understanding this, and returning to FIG. 1B, it can be appreciated that according to certain embodiments, the sphere's surface is far enough from battery 102 and LEDs 103 to thermally isolating them from the liquid nitrogen. In embodiments where the sphere surface is made of a liquid-nitrogen resistant material, the sphere is optionally not coated with any coating material.

Optionally, the LEDs and battery are stabilized inside the radiating element in a way that keeps between each of them and the sphere's outer surface large enough for thermal isolation. This is optionally achieved by holders, for example, holders 108 illustrated in FIG. 1B, which are made of a low heat conductivity material. Optionally, holders 108 are long enough to protect the LEDs and battery from the low temperature outside of the sphere. Optionally, the holders are made of a material which is UV transparent, so as not to interfere with the sterilization treatment.

In some embodiments, the radiating element is protected from other hazards than low temperature, for example, in some embodiments the spheres are coated with a coating that is particularly resistant to extremely acidic conditions.

Additionally or alternatively, the radiating elements contain heat sinks, capable of removing from the radiating element heat, for example, heat produced in the element due to the radiation of the radiation source. In some embodiments, holders 108 are made to remove heat from inside the radiating element, so as to cool the radiating element. The length of the holders is optionally adapted to allow efficient heat removal from inside the radiating element.

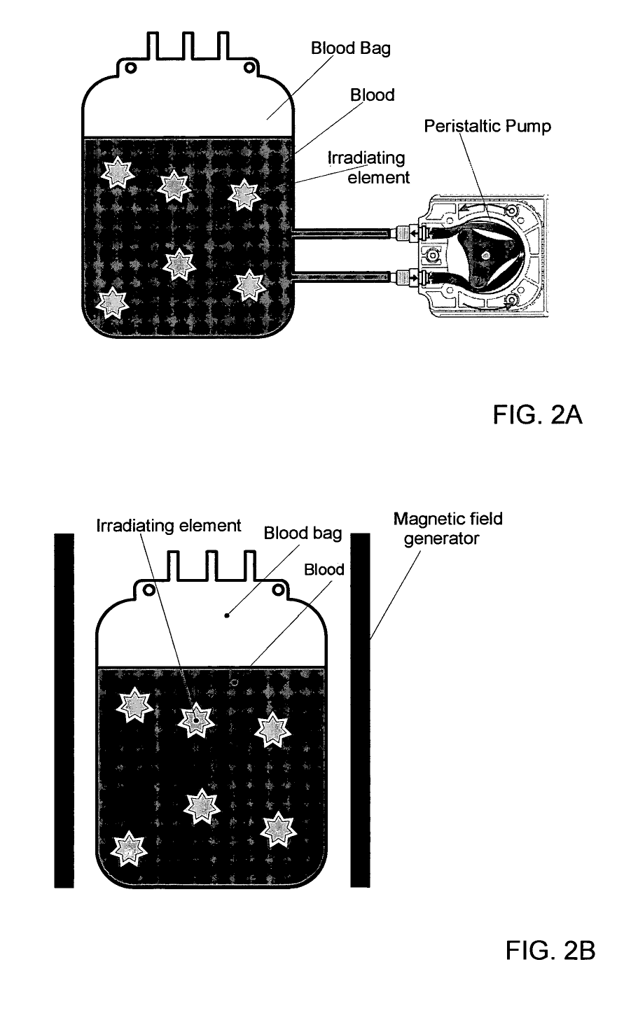

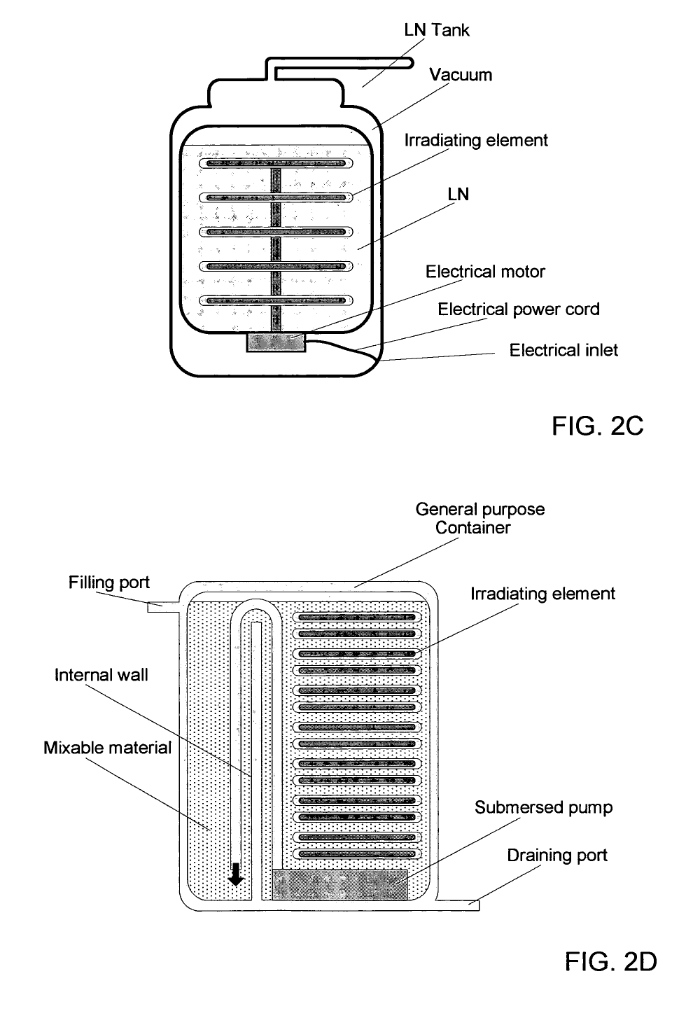

FIGS. 2A-2D provide several examples of containers that can be used with different embodiments of the invention.

In FIG. 2A a blood bag, forming a closed system, is illustrated. Optionally, the radiation occurs within a limited duration of time (e.g., up to 24 Hr). The star-like shapes illustrated inside the container represent radiating elements operating according to an exemplary embodiment. According to the example, the radiating elements are powered by an internal battery, or by an external (wireless) source, and they are activated when the blood bag is filled with a certain amount of blood.

The radiation process may take place inside a refrigerator (under hypothermia conditions) or at any other temperature, for example, room temperature, depending, for instance, on the time allowed for the process.

FIG. 2B illustrates an embodiment wherein the radiating elements are stirred with a magnetic field. In the figure, the container is positioned between two metal walls generating magnetic field that is used for steering the radiating elements. Optionally, the magnetic field's intensity and/or direction is changed in time.

FIG. 2C illustrates a liquid nitrogen (LN) sterilization container. The LN container contains a radiating element, which is powered by an external power source which supplies the energy for the radiation and the energy for the mobility of the radiating elements.

The LN container is optionally a conventional vacuum isolated container with a radiating unit installed inside.

It is noted that the radiating element illustrated in FIG. 2C is one example of a radiating element whose shape is not a sphere.

FIG. 2D depicts a general purpose radiating container, according to certain embodiments of the invention. This container enables, from one hand, a very high ratio between the radiating elements' surface and the mixable material volume, and on the other hand, the circulation enables high probability to treat large quantity of the mixable material particles in a unit of time.

### Exemplary Applications

There are many examples for systems that can benefit from radiation treatment of mixable materials, and various embodiments may be utilized in each of these examples. Some embodiments of the invention are particularly beneficial for treating mixable material by radiation that has only a short penetration depth in the mixable material. However, due to the short penetration depth and the amounts of liquid requiring purification, UV purification of large amounts of liquids using state of the art methods is many times infeasible.

Some exemplary embodiments are useful in purifying amixable material with UV radiation. Two out of many examples of mixable materials suitable for purification with UV according to some embodiments, are drinking water and light drinks.

Another example of a system that can benefit from embodiments of the present invention is blood sterilization and/or purification. As part of the effort to reduce transmission of viral diseases by blood products, donors are routinely screened for various antigens and antibodies. Screening procedures are very expensive and are not accurate. In addition, not all diseases can be screened for. Hence it would be desired to sterilize a blood unit before it is given to a patient, possibly even before the blood unit is frozen for preservation until it is required.

Furthermore, it is sometimes desired to reduce the number of white blood cells in a blood unit, or even to diminish them therefrom. Filtration might be problematic as in the methods used today it can't clean a satisfactory percentage of those white cells from the blood. In some embodiments, white blood cells are destroyed by γ radiation.

Another known problem is the problem of drinking water in places where there is no regular supply of clean water, for example, in the absence of running water, or in some wherein water supply cannot be trusted. A good example for lack of running water is water in army flasks, or when a water source is used by many people, with no awareness of sanitary rules.

An additional known problem is the problem of standing water in containers, water reservoirs, flower vases, open water bottles, swimming pools etc. Microorganisms replicate very fast inside a container and after a specific time the water must be replaced.

Another system that can benefit from some embodiments of the present invention is pasteurization of milk, juices and others. Today the pasteurization is done through heating, therefore partially cooking the substance.

### An Exemplary Treating Method

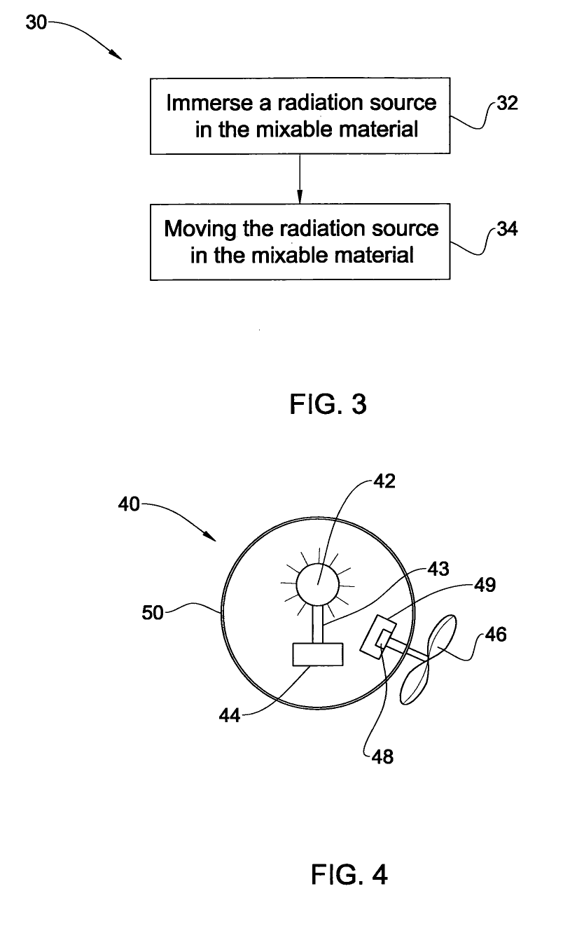

FIG. 3 is a flowchart of actions taken in one exemplary method (30) for treating mixable material with radiation in accordance with an embodiment of the invention. In this example, the method comprises immersing (32) a radiation source in the mixable material; and moving (34) the immersed radiation source inside the mixable material in a non regular fashion. Alternatively or additionally, the method comprises moving the mixable material in respect of the radiation source in a non regular fashion.

Moving in a non regular fashion is optionally achieved by moving in changing rates and/or in changing directions.

In one example, non regular motion of liquid is achieved by stirring the liquid with a stirrer that changes the stirring speed in a non regular manner. For instance, increasing and decreasing the speed with no apparent pattern.

In one example, non regular motion of a radiating element is achieved by moving the element in straight lines having different directions and/or lengths, such that directions and/or lengths do not show any apparent pattern.

In one example, a radiating element is mounted on a rotating arm, which is fixed at some point outside of the mixable material, and rotates in changing speeds. The changing rotation speeds cases the radiating element to rotate in changing radii. Optionally, the point at which the arm is fixed is moving, for example, up and down, so as to bring the radiating element to more portions of the mixable material. Optionally, the up and down movement is also non regular.

In some embodiments, non regular movement has the advantage of bringing a lot of mixable material in contact with the radiating element with a relatively low “risk” of trapping material in regular flows that will prevent it from reaching the radiation source.

In some embodiments, non regular motion of the radiating element is random, in the sense that the correlation found between the position of the radiating element and the time at which said position is observed is statistically insignificant. Optionally, the statistical significance is tested at a p value of 0.05.

In some exemplary embodiments, the movement is powered by a power source that is associated with the radiation source, such that the power source moves with the radiation source. In some such embodiments, wires connecting the radiation source to the power source and might entangle with the movement are omitted.

In some exemplary embodiments, moving the radiation source comprises moving a member mechanically attached to the radiation source. In some embodiments, the member is a paramagnetic portion, and moving the member comprises creating a magnetic field with appropriate size and direction so as to move the paramagnetic portion, and with it the radiation source.

In some exemplary embodiments, the member is a string, which connects the radiation source to a rotating arm, which is optionally outside the mixable material.

In some embodiments, the member is a propeller associated with the radiation source.

In some embodiments, the method comprises immersing and moving a plurality of radiation sources in the mixable material. Optionally, the method comprises moving the radiation sources such that the distance between each two of them changes in time. Optionally, this change is non regular. Alternatively or additionally, this change is non-periodic.

### An Exemplary Radiating Element

FIG. 4 is a schematic illustration of a radiating element (40) for treating a mixable material according to an exemplary embodiment. Radiating element 40 is shown to include a radiation source (42) electrically connected to a first power source (44) through electric wires (43). Power source 42 optionally powers radiation source 42 to emit UV radiation. Element 40 also includes a mobility agent (46) for moving radiating element 40.

Optionally, radiating element 40 also comprises a second power source (48), powering mobility agent 46. Alternatively or additionally, power source 44 powers both radiation source 42 and mobility agent 46, and the element comprises wires as required.

Optionally, radiating element 40 comprises a controller 49, for controlling the operation of mobility agent 42. Controller 49 optionally comprises switch for turning mobility agent on and off, and/or steering wheal, for controlling the direction at which element 40 moves. Optionally, controller 40 controls a processor that produces pseudo-random numbers, and controls mobility agent 46 to move element 40 in different directions for different distances and/or in different velocities in accordance with the generated random numbers.

Optionally, radiation source 42 includes one or more LEDs, optionally, UV LEDs.

In the depict embodiment, mobility agent 46 comprises a propeller. Optionally, the propeller is controlled by a processor (not shown), which turns the propeller on and off in a pseudo-random fashion, so as to form irregular movement of element 40 in the mixable material.

In some embodiments, radiation source 40 is encased in a case 50. Optionally, power source 44 and/or power source 48 are also encased in case 50.

Preferably, casing 50 has a window transparent to radiation provided by radiation source 42. Optionally, the entire casing 50 is transparent to radiation provided by radiation source 42.

In some embodiments, where radiating element 40 is adapted for radiating water or similar liquids, case 50 is waterproof. Examples of liquids which may be considered similar to water in the present context include fruit juices, milk, and blood.

In some embodiments, where radiating element 40 is adapted for radiating fluids at cryogenic temperatures, case 50 comprises a thermal insulator. In one example, case 50 comprises a double-wall casing with vacuum between the two walls, so as to thermally protect radiation source 42 and/or power source 44 from the temperature of the mixable liquid to be treated.

In operation, radiating element 40 is surrounded by the mixable material to be treated (not shown in FIG. 4). Optionally, radiating element 40 is in continuous mechanical contact only with said mixable material. This does not preclude touching from time to time walls or floor of a container holding the mixable material, touching other radiating elements inside the mixable material, or the like.

### Exemplary Systems

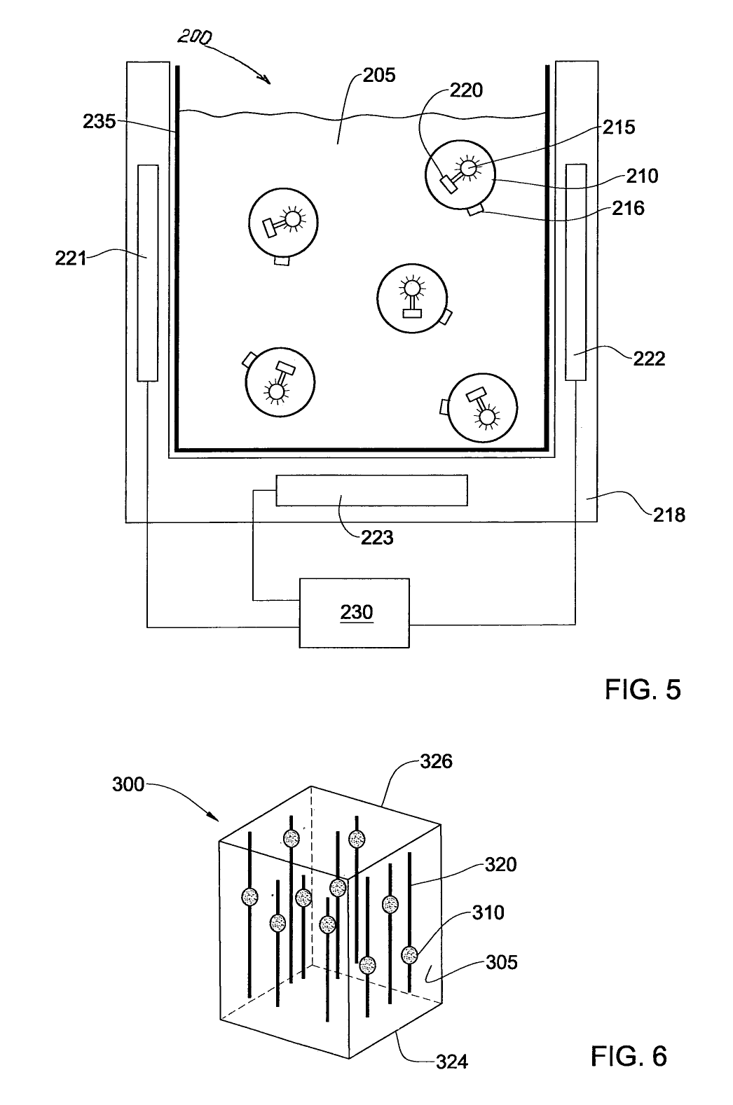

FIG. 5 is a schematic illustration of a system 200 for treating a mixable material (205) according to an exemplary embodiment. System 200 comprises at least one radiating element 210 (five are shown in the figure); and a mobility agent for moving radiating elements 210 in mixable material 205. Each of radiating elements 210 comprises a radiation source 215; and a power source 220 for powering the radiation source. In some embodiments, radiation source 215 does not require powering, for instance, in case the radiation source is a source of γ radiation or other nuclear radiation, and power source 220 is omitted.

Optionally, the mobility agent is capable of moving the radiating elements when the radiating elements are in continuous mechanical contact only with mixable material 205, and no contact with walls, floor, or any moving member is required. One kind of such mobility agent is depicted in FIG. 4 as mobility agent 46. Optionally, each of radiating elements 210 is similar to radiating element 40 of FIG. 4.

The mobility agent depicted in FIG. 5 comprises a piece of paramagnetic substance (216) in each of radiating elements 210 and a magnetic field generator 218. Optionally, magnetic field generator 218 comprises one or more electromagnets (221, 222, 223). Each of radiating elements 210 moves in accordance with the magnetic field present at the location of the paramagnetic substance 216 associated therewith. Optionally, the system comprises a controller (230), which controls magnetic field generator 218 to generate magnetic field that moves elements 210 in a non regular fashion. Optionally, the distance between each two radiating elements also changes in a non regular fashion.

Optionally, system 200 comprises a container (235) for holding mixable material 205. In one embodiment, field generator 218 is external to container 235. In some embodiments, the field generator is inside the container, optionally inside the mixable material.

Optionally, the volume occupied by the radiating elements 210 is much smaller than the volume of container 235 or of the treated mixable material (205). Optionally, the total weight of all the radiating elements together is much smaller than the weight of the mixable material to be treated. In such cases, it is advantageous to move the radiating elements inside the mixable material over moving the mixable material, because the mobility agent needs much less power for moving the small, light, emitters than for moving the mixable material. Thus, while some embodiments include means for stirring, agitating, or otherwise moving the mixable material, some embodiments do not use such means, but only means for moving the radiating elements (although, naturally, some motion in the treated material is usually caused by the motion of the radiating elements).

In some embodiments, there may be a need to take elements 210 out of mixable material 205. Optionally, this is done with a separator, separating the radiating elements from the mixable material. In one embodiment the separator is a mesh (not shown) covering an outlet (not shown), such that when the mixable material flows through the outlet, the radiating elements are stopped at the mesh. In one embodiment, the separator comprises a rod with a strong magnet, and when the rod is moved in the mixable material and passes nearby a radiating element, the magnetic portion 216 is attached to the magnetic rod. When the rod is taken out of the mixable material, it takes with it the radiating elements.

FIG. 6 is a schematic illustration of a system 300 for treating a mixable material (305) according to an exemplary embodiment. System 300 comprises a plurality radiating element 310 moving along chords 320, that go between the floor 324 and the ceiling 326 of a container holding the mixable material. Optionally, chords 320 also move in trajectories parallel to floor 324, while being kept stretched between the ceiling and the floor. Optionally, the chords move randomly, for example, perform “random walk”, taking steps of constant length in random directions. Optionally, each step is of random length. Alternatively or additionally, the up and down movement of the radiating elements along the chords is random, for instance, each element performs one-dimensional random walk along the chord it is associated with. The decision about taking a move of a radiation source is optionally taken at the radiation source itself (for instance, by a processor associated therewith), or away of the radiating element, for instance, at a central control residing outside the mixable material. In the latter case, control signals are optionally transmitted wirelessly from the central control to the radiating elements. Similarly, a motor moving the wires can include a processor for determining the movement direction of each of the wires, or such decisions are taken away, for instance, in a central control, and transmitted to the motor.

### General Comments

The terms “comprises”, “comprising”, “includes”, “including”, “having” and their conjugates mean “including but not limited to”.

The term “consisting of means “including and limited to”.

As used herein, the singular form “a”, “an” and “the” include plural references unless the context clearly dictates otherwise. For example, the term “a radiating element” or “at least one radiating element” may include a plurality of elements, and is used to disclose both embodiments of one element and embodiments of more than one element.

The term “exemplary” is used herein to mean serving as an example, instance, or illustration, and not necessarily as deserving imitation due to excellence.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

The present invention has been described with a certain degree of particularity, but those versed in the art will readily appreciate that various alterations and modifications may be carried out, without departing from the scope of the following claims:

1. A method of irradiating a mixable material, the method comprising:
immersing a radiation source in the mixable material; and
providing relative displacement between the immersed radiation source and the mixable material in a non regular fashion.

2. A method according to claim 1, wherein providing relative displacement comprises powering displacement of the radiation source by a power source that moves with the radiation source.

3-4. (canceled)

5. A method according to claim 1, wherein providing relative displacement in a non regular fashion comprises creating a plurality of movements, each having a direction and distance, wherein at least one of the direction and the distance of each of said movements is independent of the direction and the distance of the preceding movement.

6-7. (canceled)

8. A method according to claim 1, comprising:
immersing a plurality of radiation sources in the mixable material; and
providing relative displacement between each of said plurality of radiation sources and the mixable material in a non regular fashion.

9-11. (canceled)

12. A method according to claim 1, comprising powering said radiation source with a power source that moves with the radiation source.

13. A method according to claim 1, wherein said radiation source is passive.

14. A radiating element configured for immersing in a mixable material, comprising:
a radiation source; and
a mobility agent adapted for moving the radiating element in a non regular fashion, for generating relative mobility of the radiating element in the mixable material.

15-19. (canceled)

20. A radiating element according to claim 14, wherein said radiation source comprises a UV radiation source.

21. A radiating element according to claim 14, comprising a processor that generates pseudo-random numbers and uses said numbers for controlling the movement of the radiating element.

22. A radiating element comprising:
a passive radiation source, and
a mobility agent adapted for moving the radiating element.

23. (canceled)

26. A radiating element according to claim 22, comprising a processor that generates pseudo-random numbers and uses said numbers for controlling the movement of the radiating element.

27. A system for treating a mixable material, the system comprising:
at least one radiating element; and
a mobility agent for moving the at least one radiating element in a non-regular fashion, generating relative mobility of the radiating element in the mixable material.

28. (canceled)

29. A system according to claim 27, comprising a field generator for coupling a force field with said at least one radiating element.

30. (canceled)

31. A system according to claim 27, wherein said at least one radiating element comprises a plurality of radiating elements, the distance between each two of said plurality of radiating elements changes in a non regular fashion.

32-34. (canceled)

35. A system according to claim 27, comprising a container for holding the mixable material, wherein said container has a volume that is at least 10 times larger than the volume of all said at least one radiating elements together.

36. (canceled)

37. A method according to claim 1, wherein providing relative displacement includes:
processing pseudo-random numbers; and
controlling at least one of direction, distance and velocity of the radiation source in accordance with the pseudo-random numbers.

38. The radiating element according to claim 14, controllable by an external controller.

39. A method of irradiating a mixable material, the method comprising:
immersing a radiating element in the mixable material;
generating relative mobility of the radiating element in the mixable material;
determining treatment level parameters; and
changing treatment parameters in response to determined treatment level parameters.

40. The method according to claim 39, wherein determining the treatment level parameters is done during operation.

41. The method according to claim 40, wherein determining the treatment level parameters during operation is done by measuring.

42. The method according to claim 39, further comprising:
changing radiation intensity upon achieving a satisfactory level of treatment.
